(19) United States
(12) Patent Application Publication
(10) Pub. No.: US 2021/0060515 A1
(43) Pub. Date: Mar. 4, 2021

(54) SUBSTRATE FOR PROTEIN PRINTING

(71) Applicants: ALVEOLE, Paris (FR); Centre National de la Recherche Scientique, Paris (FR); Université de Bordeaux, Bordeaux (FR)

(72) Inventors: Vincent Studer, Bordeaux (FR); Louise Bonnemay, Paris (FR)

(73) Assignees: ALVEOLE, Paris (FR); Centre National de la Recherche Scientique, Paris (FR); Université de Bordeaux, Bordeaux (FR)

(21) Appl. No.: 16/982,306

(22) PCT Filed: Mar. 19, 2019
(86) PCT No.: PCT/EP2019/056849

§ 371 Date: Nov. 5, 2020

(30) Foreign Application Priority Data
Mar. 20, 2018 (FR) 1852391

(51) Int. Cl. B01J 19/00; C07K 1/04

(52) U.S. Cl. CPC B01J 19/0046; B01J 2219/00711; C07K 1/045

(57) ABSTRACT

Product for printing proteins comprising a substrate (1), a nanoscale polymer first layer (3), which is nonstick for the proteins, deposited on the substrate (1), and a second layer of a benzophenone (2), deposited on the first layer (3), wherein the second layer (2) is solid and soluble in a solvent, and the first layer (3) is insoluble in the solvent.

### TECHNICAL FIELD

The present application relates to the general field of grafting proteins onto a substrate and, in particular, the grafting of proteins in a predefined pattern onto a substrate via an optical means.

### BACKGROUND

The international application published under the number WO 2016/050980 (referred to hereinbelow as the “Studer publication”), relates to a process for the microstructured grafting, or grafting in a pattern, of proteins onto a printing or photochemical substrate, in which the substrate is covered with a nanoscale (between 1 nm and 20 nm) antifouling layer, i.e. a layer that is nonstick for living cells. This type of nonstick layer is particularly a polymer brush or brush of a polymer and in particular a brush of a PEG (polyethylene glycol). A layer that is nonstick for proteins is intended to be bought into contact with solutions of proteins, solutions which are necessarily aqueous in this known process, and is therefore insoluble in water insofar as is necessary for the use thereof. Such a layer is also intended to be illuminated by radiation in the absorption spectrum of benzophenone (between 300 nm and 400 nm) and is therefore resistant to this radiation, insofar as is necessary for the printing thereof.

The process of the Studer publication essentially consists in bringing into contact with, or depositing on, a substrate surface-treated with a PEG brush, a drop of an aqueous solution of a benzophenone, then in illuminating the nanoscale layer of the brush, in the presence of the drop, with radiation having a wavelength within the absorption spectrum of benzophenone (between 300 nm and 400 nm) according to a predefined pattern. After rinsing of the benzophenone in solution, the substrate obtained is selectively adhesive for proteins in the illuminated zones; it thus enables the printing or deposition of proteins and then of cells on the substrate and the multiplication thereof only in the zones of the pattern, i.e. according to a specific adhesion.

The benzophenone used in the Studer publication is necessarily a benzophenone that is soluble in a solvent which is water, so as to be able to be placed in the form of an aqueous solution.

However, in the process of the Studer publication, the presence of a drop of aqueous solution when illuminating the layer makes it necessary to compensate for the inevitable evaporation of the drop during the illumination time in order to stabilize the concentration of benzophenone in the aqueous solution, in order to obtain a reproducibility of the printing of a pattern of proteins on the substrate. The drying of the drop of aqueous solution of benzophenone therefore constitutes a problem in this known process. One solution could consist in providing a supply of water to the drop, via microfluidic means, to compensate for the loss of liquid caused by the drying or the evaporation of the drop, so as to keep the volume thereof constant. However, such a solution complicates the experimental device. Thus, in the process of the Studer publication, maintaining a constant concentration of benzophenone in the drop appears to be desirable but difficult.

### GENERAL PRESENTATION

In this context, the invention relates to a product for printing proteins, comprising a substrate, a nanoscale first layer of polymer, which is nonstick for the proteins, deposited on the substrate, and a solid second layer of benzophenone, deposited on the first layer. The solid second layer is soluble in a solvent, and the first layer is insoluble in the solvent.

The word “soluble” will be understood, in the present disclosure, as the property, for a solid material, of being able to be dissolved in a given solvent. The word “solvent” will be understood, in the present disclosure, as meaning a liquid capable of dissolving a solid or of dispersing the molecules or atoms thereof.

The word “layer” will be understood, in the present disclosure, as a film of material which is solid, in particular pasty or gelled, with the exception of a film of liquid material. The thickness of a layer may be either constant for a film with flat and parallel faces, or variable for a rippled or curved (in particular dome-shaped) film.

The word “deposited” will be understood, in the present disclosure, as “in mechanical contact”. For a layer of material positioned on a solid substrate, this word will denote a form of mechanical contact without relative displacement of the atoms of the material relative to the substrate or without flow and will signify “attached”, whilst for a solution of a material in a liquid, positioned on a solid substrate, this word will denote a mechanical contact with possible flow or relative displacement of the atoms of the material and of the liquid, relative to the substrate.

The word “thin” or “nanoscale” layer will be understood, in the present disclosure, as a layer having a thickness of between 1 nm and 2000 nm, without excluding layers thinner than a nanometer and that are nonstick for proteins.

In variants of the product, the following provisions are adopted, independently or combined together:
the second layer is soluble in a polar solvent;
the first layer is a polymer brush;
the substrate is a glass; the second layer is soluble in water, in ethanol or in isopropanol;
the polymer is a polyethylene glycol (PEG).

The invention also relates to a process comprising the following steps:
providing a substrate,
depositing on the substrate a nanoscale first layer of polymer that is nonstick for proteins;
depositing on the first layer a second layer of a benzophenone, the second layer being soluble in a solvent and the first layer being insoluble in said solvent.

This process makes it possible to obtain, or fabricate, a protein-printing product as described above.

In a variant of the process, the second layer is deposited on the first layer according to the following steps:
depositing, on the first layer, the benzophenone in solution in the solvent; and
evaporating the solvent.

In another variant of the process, the second layer is deposited, on the first layer, by a physical vapor deposition (PVD) of the benzophenone.

For the photoprinting, onto the first layer, of a pattern that is adhesive for proteins, the process comprises the following additional steps:
illuminating the first layer, in an absorption spectrum of benzophenone, according to the pattern;
dissolving the second layer in the solvent;
rinsing the solvent.

For the printing of the proteins, according to the pattern, onto the first layer, the process comprises the following additional steps:
depositing, on the first layer, an aqueous solution of the proteins;
rinsing the aqueous solution of the proteins.

The abovementioned features and advantages, along with others, will become apparent from reading the following detailed description of exemplary embodiments of the invention. This detailed description refers to the appended drawings. However, it should be noted that the invention is not limited to these examples.

### BRIEF DESCRIPTION OF THE DRAWINGS

The appended drawings are schematic and are not necessarily to scale; they are intended primarily to illustrate the principles of the invention.

FIG. 1 represents an example of a product for printing proteins.

### DETAILED DESCRIPTION OF EXAMPLE(S)

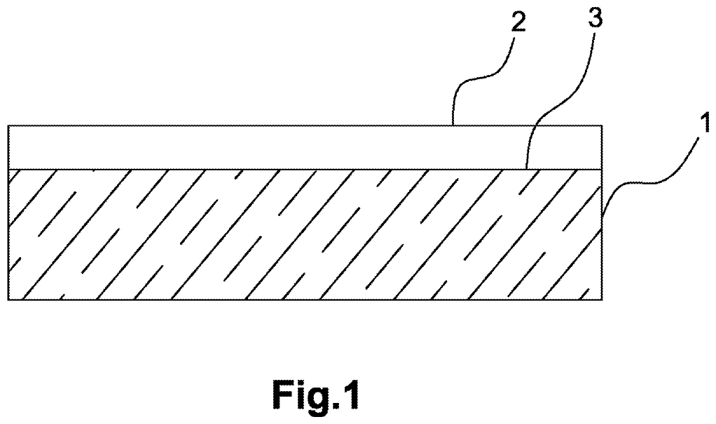

The product for printing proteins from FIG. 1 comprises:
a glass substrate 1,
a nanoscale polymer first layer 3, which is nonstick for proteins, attached to or deposited on one of the faces of the substrate 1, and
a second layer, or solid deposit, of benzophenone 2 deposited on the nonstick polymer layer 3 and soluble in a solvent.

The nonstick first layer 3 is in mechanical contact with the substrate 1 and the benzophenone layer 2, and the nonstick first layer 3 is positioned between the second layer 2 and the glass substrate 1. The substrate 1 may be flat, as shown.

In a first embodiment, the substrate 1 is, in a manner known from the prior art, covered with the polymer first layer 3 that is nonstick for living cells, or nonstick layer, or antifouling layer within the meaning of the Studer publication mentioned above. This first layer is, in this first embodiment, a polymer brush and the polymer is a PEG (polyethylene glycol). This first layer 3 is deposited on the substrate 1 by means known from the prior art.

In a first method for obtaining the product, a liquid solution of a water-soluble benzophenone is produced from a crystalline powder of the soluble benzophenone, which is not transparent in the visible spectrum in this powdery form, and deionized water. The soluble benzophenone has, for example, the chemical formula: (4-benzoylbenzyl)trimethylammonium chloride.

Next, one drop or several drops of the solution is/are deposited on the first layer 3 until the liquid solution has spread out over the substrate, i.e. over the substrate covered with the first layer, in order to obtain, on the surface thereof, a film of solution, with parallel or rippled or curved faces.

The water is then evaporated from the solution. For this, it is possible to stove the system obtained, for example at 70° C. or let it dry naturally at room temperature, in order to dry out the solution by evaporation. The same method would be applied for a solvent other than water, provided that the solvent used is compatible with the first layer 3. Thus, after drying, a more or less hard second layer 2 of transparent, i.e. noncrystalline, benzophenone is obtained. It should be noted that a person skilled in the art would expect to reobtain the crystalline, and therefore non-transparent, benzophenone powder, separated from the first layer 3 and from the substrate. However, for this type of benzophenone, surprisingly, the benzophenone remains in solid form in a homogeneous layer that adheres to the substrate and is optically transparent, noncrystalline, probably in the form of an amorphous solid. The consistency of this second layer and the thickness thereof make it possible in particular to scratch it in a durable manner.

Generally, any benzophenone having, once deposited as a layer, a transparence in the visible spectrum or a noncrystallization, is in accordance with the teaching of the present disclosure and can therefore be used within the frame of the invention. The layer may be obtained by evaporation of a solution of benzophenone in a solvent, or by any other method for depositing a layer of this benzophenone on the first layer 3.

Advantageously, the noncrystallization of the benzophenone layer obtained enables the photoprinting of patterns by illumination of the first layer 3, without degradation of the layer 3 due to crystals. The photoprinting is carried out with radiation in the absorption spectrum of the benzophenone, through the second layer 2 or through the substrate 1, chosen to be sufficiently transparent to the illumination radiation.

After illumination, a lighting up, for example in the visible spectrum, at low-angled incidence, of this second layer 2, conveniently reveals, at the outer surface of the second layer 2, the patterns which have been imaged on the first layer 3, positioned on the inner surface of the second layer 2, without having need to access the first layer 3.

The photoprinting of patterns is thus durable and recognizable to the naked eye, at the surface of the second layer 2, which makes it possible to easily distinguish a photoprinted layer from a non-photoprinted layer.

In a known manner, the radiation used to illuminate the patterns will have a wavelength or a spectrum located in the absorption band of benzophenone, which lies between 300 nm and 400 nm.

The layer may be scratched in order to measure the thickness thereof and layers of greater than 100 microns may be obtained easily. It is also possible to control the initial amount of benzophenone solution in order to obtain a controlled layer thickness. A person skilled in the art will be able to determine in each case the thinnest layer that it is possible to achieve by simple execution operations.

It should be noted that reducing the thickness of the layer makes it possible to prevent or minimize interferences of the radiation between the faces of the layers, and pattern printing errors. It is also possible to use mixtures of solvents to homogenize the spread of the layer, these solvents then being evacuated. The product, once the benzophenone layer is obtained, can be stored and transported easily with no particular precautions. It can be exposed to light on an optical system without microfluidic or fluidic means, for opposing the drying or evaporation of a drop of aqueous benzophenone solution, which would be necessary in the process of the Studer publication, to obtain a constant concentration of benzophenone on top of the first layer during the illumination, in order to also obtain a controlled subsequent adhesion for the proteins, in the illuminated zones.

This advantage is obtained owing to the benzophenone second layer 2, which is solid (e.g. pasty or gelled) and in which the benzophenone concentration is more stable, on the timescale of the illumination, than in a drop of liquid benzophenone solution.

It will be noted that a person skilled in the art would not use, in the prior art and the abovementioned Studer publication, a solvent more volatile than water, in order not to accentuate the problem of drying of the drop of aqueous benzophenone solution during the illumination.

The product can also be transported after photoprinting in order to be rinsed in a clean room by dissolving the second layer in a suitable solvent.

This solvent may be a deionized water but it has been found that ethanol or isopropanol, which are polar solvents, are well suited to the invention. A benzophenone soluble in a polar solvent will therefore be particularly suitable for the invention.

After rinsing, the nonstick first layer, rendered adhesive for the proteins, according to the patterns, owing to the illumination, will be able to be brought into contact with a solution of proteins in order to obtain a pattern of proteins printed on the first layer, according to the illuminated patterns.

A water-insoluble benzophenone could also be used if a solvent is found in which no crystallization is observed on drying the layer. Benzoin ethyl ether could thus be used when using acetone as solvent.

In a second embodiment, the second layer is deposited in a better controlled manner in terms of thickness in a PVD (physical vapor deposition) rack or by any technique (PVD, CVD, etc.) that makes it possible to deposit a transparent (noncrystalline) benzophenone layer on a substrate, without destroying the nonstick layer.

The physical vapor deposition will make it possible to produce thin layers of benzophenone, having a thickness that is very even and that therefore leads to less interferences during the photoprinting. This deposition method is thus particularly advantageous.

This method is preferred for thin layers having a thickness of less than 1000 nm or submicron thickness, which may be difficult to obtain by drying, at least without crystallization, for a particular benzophenone.

A benzophenone suitable for this type of vapor-phase submicron deposition will be, for example, a soluble benzophenone of sulisobenzone type or benzophenone-4 type or a benzophenone of (4-benzoylbenzyl)trimethylammonium chloride type.

For an unknown benzophenone to be deposited as a thin layer having a given thickness, when the observation of the dried layer will be possible optically, a person skilled in the art could observe whether it is noncrystalline and particularly whether it is homogeneous and transparent, in order to determine whether the unknown benzophenone is well suited to the invention. In the event of crystallization, a person skilled in the art could carry out the deposition in the vapor phase.

The teaching of the application therefore extends to benzophenones which do not crystallize as thin layers during a deposition of given thickness. This criterion could be used to choose a suitable deposition process for an unknown benzophenone or to obtain a new thickness for a known benzophenone. A person skilled in the art could thus firstly use the drying of a drop of benzophenone solution, then the deposition by thin layer techniques in the vapor phase to determine what benzophenone layer thickness range is attainable according to the invention for a given benzophenone.

This combination of deposition of thin layers in the liquid phase or in the vapor phase makes it possible to potentially produce a considerable range of thicknesses of benzophenone layers, for an arbitrary benzophenone compatible with the photoprinting of proteins onto a nonstick layer of nanoscale thickness deposited on a substrate.

Any method for depositing thin layers known from the prior art and compatible with the deposition of a layer of soluble benzophenone that is the most even and of controlled thickness in the face of the illumination wavelength could be used to produce the product of the invention.

The solvent used for the rinsing, i.e. the dissolving, of the second layer could be any solvent provided that it is compatible with the substrate and the nonstick layer, in particular the nonstick layer will be insoluble in the solvent as will the substrate. For the biocompatibility thereof with living cells, water will be the preferred solvent for the rinsing operation, the layers that are nonstick for proteins and the glass generally used as substrate being water-resistant.

It should be noted that the operation for depositing a solid layer, in particular in the form of gel, increases the concentration of benzophenone relative to a liquid and that the photoprinting time, all other things being equal, is thereby shortened. Thus, for a pattern printing time of 40 seconds with a drop of aqueous solution in contact with the first layer, a printing time of 0.5 second is easily obtained with a second layer obtained by evaporation of the drop according to the present application.

Furthermore, knowing that oxygen or dioxygen is involved in the mechanism for rendering the illuminated zones adhesive for proteins, the deposition of a benzophenone layer makes it possible to have a better replenishment of dioxygen at the first layer 3 than with a drop of aqueous solution, which is thicker and therefore less permeable to oxygen than the second layer 2, and to improve the homogeneity and the instantaneous reproducibility of the photoprinting.

It should also be noted that the deposited layer of benzophenone deposited according to the invention by drying or CVD or PVD has a stable concentration, which improves the long-term reproducibility of the printing of proteins on the nonstick layer of the substrate.

The invention extends to any transparent or noncrystalline solid deposit of benzophenone, deposited on a layer that is nonstick for proteins. Specifically, in a third embodiment, the benzophenone layer may be deposited in the form of a transparent heap, without seeking to immediately give it a substantially uniform thickness, for example by depositing a drop of benzophenone solution with a pipette, in order to obtain a film having a typical thickness of 100 microns and a variable, substantially circular, in particular curved or dome-shaped, shape by drying the drop without spreading, thus retaining the transparency or noncrystallization.

In order to use the above deposit, a drop of solvent will be deposited on the solid deposit or heap above in order to dissolve it and the reformed solution will be respread over the first layer 3. After evaporation of the solvent, a noncrystalline transparent layer reappears.

In the case of aging, over time, of an initially spread benzophenone layer, deposited according to this third embodiment, it is noted that it is subsequently possible to deposit a drop of solvent on the heap or the layer, in order to reform a solution and to dry it in the form of a thin layer of uniform thickness of the order for example of a few microns. It is thus possible to compensate for or repair, with this method, a spreading defect of the benzophenone gel as a thin layer deforming its surface on the first layer 3 that is nonstick for proteins. Generally, this method of compensation or repair by addition of a drop of solvent applies to all the embodiments of the invention, i.e. to any deposited benzophenone layer.

Finally, the teaching of the present application thus appears to extend to any transparent or nontransparent, noncrystalline benzophenone layer deposited at the surface of a layer that is nonstick for proteins, the benzophenone layer being soluble in a solvent in which the nonstick layer is insoluble.

The invention is industrially applicable or useful in the field of printing proteins on a substrate.

1. A product for printing proteins, comprising:
a substrate,
a nanoscale first layer of polymer, which is nonstick for proteins, deposited on the substrate,
characterized in that it further comprises,
a second layer of benzophenone, deposited on the first layer,
wherein the second layer is soluble in a solvent and the first layer is insoluble in the solvent.

2. The product as claimed in claim 1, wherein the second layer is soluble in a polar solvent.

3. The product as claimed in claim 1, wherein the first layer is a polymer brush.

4. The product as claimed in claim 1, wherein the substrate is made of glass.

5. The product as claimed in claim 1, wherein the second layer is soluble in water.

6. The product as claimed in claim 1, wherein the second layer is soluble in ethanol.

7. The product as claimed in claim 1, wherein the second layer is soluble in isopropanol.

8. The product as claimed in claim 3, wherein the polymer is polyethylene glycol (PEG).

9. A process for obtaining the product as claimed in claim 1 comprising the following steps:
depositing the first layer on the substrate; and
depositing the second layer on the first layer.

10. The process as claimed in claim 9, wherein the second layer is deposited on the first layer according to the following steps:
depositing, on the first layer, the benzophenone in solution in the solvent; and
evaporating the solvent.

11. The process as claimed in claim 9, wherein the second layer is deposited on the first layer by a physical vapor deposition (PVD) of the benzophenone.

12. The process as claimed in claim 9, for photoprinting onto the first layer of a pattern that is adhesive for proteins, comprising the following additional steps:
illuminating the first layer, in an absorption spectrum of benzophenone, according to the pattern;
dissolving the second layer in the solvent; and
rinsing the solvent.

13. The process as claimed in claim 12, for printing proteins onto the first layer, according to the pattern, comprising the following additional steps:
depositing, on the first layer, an aqueous solution of proteins; and
rinsing the aqueous solution of proteins.
